(19) United States
(12) Patent Application Publication
(10) Pub. No.: US 2025/0327398 A1
(43) Pub. Date: Oct. 23, 2025

(54) TRACER PARTICLES AND RELATED METHODS

(71) Applicant: Saudi Arabian Oil Company, Dhahran (SA)

(72) Inventors: Amr I. Abdel-Fattah, Dhahran (SA); Afnan Mashat, Dhahran (SA)

(21) Appl. No.: 18/639,488

(22) Filed: Apr. 18, 2024

(51) Int. Cl. E21B 47/11; E21B 47/117; G01N 33/18

(52) U.S. Cl. CPC E21B 47/111; E21B 47/117; G01N 33/18

(57) ABSTRACT

The disclosure relates to compositions and methods that include tracer particles that include precursor particles isolated from a petroleum reservoir and a plurality of tags. The tracer particles can be used for different hydrocarbon reservoir applications, including determining the volume of water-filled pores between wells in a petroleum reservoir and tracking water flow through a petroleum reservoir.

### FIELD

The disclosure relates to compositions and methods that include tracer particles that include precursor particles isolated from a petroleum reservoir and a plurality of tags. The tracer particles can be used for different hydrocarbon reservoir applications, including determining the volume of water-filled pores between wells in a petroleum reservoir and tracking water flow through a petroleum reservoir.

### BACKGROUND

The oil and gas industry uses tracer particles as a tool for tracking water flow through the reservoir and volume of water-filled pores between wells. These tracer particles are injected with water from the injector wells and monitored and analyzed at the production wells. The data collected from tracer particles offers information about the petroleum reservoir, such as the flow pathways, hydrodynamic parameters, and well connectivity, among other characteristics.

### SUMMARY

The disclosure relates to compositions and methods that include tracer particles that include precursor particles isolated from a petroleum reservoir and a plurality of tags. The tracer particles can be used for different hydrocarbon reservoir applications, including determining the volume of water-filled pores between wells in a petroleum reservoir and tracking water flow through a petroleum reservoir.

Without wishing to be bound by theory, it is believed that the use of tracer particles comprising precursor particles isolated from a petroleum reservoir may be more stable than synthetic tracer particles because, for example, the tracer particles comprising precursor particles are known to be relatively stable under the conditions in the petroleum reservoir.

Without wishing to be bound by theory, it is believed that tracer particles comprising precursor particles isolated from a petroleum reservoir may move more freely through a petroleum reservoir than synthetic tracer particles because, for example, the tracer particles comprising precursor particles are known to be relatively transportable under the conditions in the petroleum reservoir.

The tracer particles comprising precursor particles isolated from a petroleum reservoir may be more stable than synthetic tracer particles under the conditions in the petroleum reservoir. The tracer particles comprising precursor particles isolated from a petroleum reservoir may be more stable than synthetic tracer particles under high temperature conditions. The tracer particles comprising precursor particles isolated from a petroleum reservoir may be more stable than synthetic tracer particles under high salinity conditions. The tracer particles comprising precursor particles isolated from a petroleum reservoir may be more stable than synthetic tracer particles under high temperature and salinity conditions.

The tracer particles comprising precursor particles isolated from a petroleum reservoir may move more freely through a petroleum reservoir than synthetic tracer particles under the conditions in the petroleum reservoir. The tracer particles comprising precursor particles isolated from a petroleum reservoir may move more freely through a petroleum reservoir than synthetic tracer particles under high temperature conditions. The tracer particles comprising precursor particles isolated from a petroleum reservoir may move more freely through a petroleum reservoir than synthetic tracer particles under high salinity conditions. The tracer particles comprising precursor particles isolated from a petroleum reservoir may move more freely through a petroleum reservoir than synthetic tracer particles under high temperature and salinity conditions.

The methods may involve fewer processing steps to prepare tracer particles relative to certain other methods.

The methods may involve fewer steps to determine the volume of water-filled pores between wells in a petroleum reservoir relative to certain other methods.

The methods may involve fewer steps to track water flow through a petroleum reservoir relative to certain other methods.

In a first aspect, the disclosure provides a method, comprising: isolating a precursor particle from a petroleum reservoir; labeling the precursor particle with a plurality of tags to form a tracer particle; dispersing a plurality of tracer particles in injection water to form a tracer particle solution; injecting the tracer particle solution into an injection well of a petroleum reservoir; collecting a sample of produced water from one or more producing wells; and measuring the concentration of tracer particles from the one or more samples of produced water.

In some embodiments, the precursor particle is isolated from produced water from a petroleum reservoir.

In some embodiments, the precursor particle is isolated from the petroleum reservoir by ultrafiltration or cross-flow filtration.

In some embodiments, the precursor particle comprises at least one member selected from clay and mineral fragments.

In some embodiments, the surface of the tracer particle comprises at least one member selected from a polyethylene glycol (PEG), an antibody, a peptide, an organic dye, a paramagnetic functional group, and a super magnetic functional group.

In some embodiments, the organic dye induces the fluorescent properties of the tracer particle. In some embodiments, the organic dye enhances the fluorescent properties of the tracer particle.

In some embodiments, the surface of the tracer particle comprises at least one member selected from an amino group (—NH₂), an alkylamino group (—NHR), a dialkylamino group (—NR₂), a carboxy group (—COOH), a hydroxy group (—OH), a thiol group (—SH), a thioether group (—SR), an aldehyde group (—CHO), an azide group (—N₃), and an alkyne group (—CH), wherein each R is C₁₋₁₂ alkyl.

In some embodiments, the tracer particle has a diameter of from about 1 nm to about 1000 nm. In some embodiments, the tracer particle has a diameter of from about 1 nm to about 100 nm. In some embodiments, the plurality of tags comprise at least one member selected from a chemical tag and a nanoparticle tag. In some embodiments, the plurality of tags comprise at least one chemical tag. In some embodiments, the plurality of tags comprise at least one nanoparticle tag.

In some embodiments, the plurality of tags comprise at least one member selected from a chemical fluorescent tag, a nanoparticle fluorescent tag, a chemical magnetic tag, a nanoparticle magnetic tag, a chemical acoustic contrast tag, a nanoparticle acoustic contrast tag, a chemical thermal tag, a nanoparticle thermal tag, a chemical radioactive tag, and a nanoparticle radioactive tag.

In some embodiments, the plurality of tags comprise at least one fluorescent tag. In some embodiments, the plurality of tags comprise at least one chemical fluorescent tag. In some embodiments, the plurality of tags comprise at least one nanoparticle fluorescent tag.

In some embodiments, the plurality of tags comprise at least one magnetic tag. In some embodiments, the plurality of tags comprise at least one chemical magnetic tag. In some embodiments, the plurality of tags comprise at least one nanoparticle magnetic tag.

In some embodiments, the plurality of tags comprise at least one acoustic contrast tag. In some embodiments, the plurality of tags comprise at least one chemical acoustic contrast tag. In some embodiments, the plurality of tags comprise at least one nanoparticle acoustic contrast tag.

In some embodiments, the plurality of tags comprise at least one thermal tag. In some embodiments, the plurality of tags comprise at least one chemical thermal tag. In some embodiments, the plurality of tags comprise at least one nanoparticle thermal tag.

In some embodiments, the plurality of tags comprise at least one radioactive tag. In some embodiments, the plurality of tags comprise at least one chemical radioactive tag. In some embodiments, the plurality of tags comprise at least one nanoparticle radioactive tag.

In some embodiments, the plurality of tags are bonded to the precursor particle through chemisorption or physisorption.

In some embodiments, the plurality of tags are bonded to the precursor particle through chemisorption, wherein the chemisorption is an ionic bond or a covalent bond.

In some embodiments, the plurality of tags are bonded to the precursor particle through physisorption, wherein the physisorption is Van der Waals forces or dipole-dipole interaction.

In some embodiments, about 10% or more of the surface area of the tracer particle is covered by the plurality of tags. In some embodiments, about 20% or more of the surface area of the tracer particle is covered by the plurality of tags.

In some embodiments, the tracer particle further comprises a surfactant. In some embodiments, the surfactant comprises at least one member selected from a nonionic surfactant, a cationic surfactant, an anionic surfactant, and a zwitterionic surfactant.

In some embodiments, the surfactant comprises at least one member selected from glyceryl monostearate (GMS), sorbitan monostearate (Span 60), poloxamer 188 (Pluronic F68), polysorbate 80 (Tween 80), cetyltrimethylammonium bromide (CTAB), didodecyldimethylammonium bromide (DDAB), sodium dodecyl sulfate (SDS), sodium cholate (SC), oleyl amidopropyl betaine (OAPB), and 3-(N,N-dimethyltetradecylammonio) propane-1-sulfonate (SB3-14).

In some embodiments, a concentration of about 50 parts per trillion of tracer particles can be detected from the one or more samples of produced water.

In some embodiments, the method further comprises plotting the concentration of tracer particles from the one or more samples of produced water as a function of transport time.

In some embodiments, the method further comprises determining the volume of water-filled pores between wells in a petroleum reservoir.

In some embodiments, the method further comprises tracking water flow through a petroleum reservoir.

### DESCRIPTION OF THE FIGURES

FIG. 1 illustrates tracer particles comprising precursor particles isolated from a petroleum reservoir with synthetic tracer particles.

FIG. 2 schematically illustrates an exemplary process of isolating precursor particles from a petroleum reservoir; labeling the precursor particle with a plurality of tags; injecting the tracer particles into an injection well of a petroleum reservoir; and collecting a sample of produced water from one or more producing wells.

### DETAILED DESCRIPTION

The disclosed method comprises isolating a precursor particle from a petroleum reservoir; labeling the precursor particle with a plurality of tags to form a tracer particle; dispersing a plurality of tracer particles in injection water to form a tracer particle solution; injecting the tracer particle solution into an injection well of a petroleum reservoir; collecting a sample of produced water from one or more producing wells; and measuring the concentration of tracer particles from the one or more samples of produced water.

In some embodiments, the precursor particle is isolated from produced water from a petroleum reservoir.

In some embodiments, the precursor particle is isolated from the petroleum reservoir by ultrafiltration or cross-flow filtration. In some embodiments, the precursor particle is isolated from the petroleum reservoir by ultrafiltration. In some embodiments, the precursor particle is isolated from the petroleum reservoir by cross-flow filtration.

In some embodiments, the precursor particle comprises at least one member selected from clay and mineral fragments. In some embodiments, the precursor particle comprises clay. In some embodiments, the precursor particle comprises mineral fragments. In some embodiments, the precursor particle comprises clay and mineral fragments.

In some embodiments, the surface of the tracer particle comprises at least one member selected from an amino group (—NH₂), an alkylamino group (—NHR), a dialkylamino group (—NR₂), a carboxy group (—COOH), a hydroxy group (—OH), a thiol group (—SH), a thioether group (—SR), an aldehyde group (—CHO), an azide group (—N₃), and an alkyne group (—CH), wherein each R is C₁₋₁₂ alkyl. In some embodiments, the surface of the tracer particle comprises an amino group (—NH₂). In some embodiments, the surface of the tracer particle comprises an alkylamino group (—NHR), wherein R is C₁₋₁₂ alkyl. In some embodiments, the surface of the tracer particle comprises a dialkylamino group (—NR₂), wherein each R is C₁₋₁₂ alkyl. In some embodiments, the surface of the tracer particle comprises a carboxy group (—COOH). In some embodiments, the surface of the tracer particle comprises a hydroxy group (—OH). In some embodiments, the surface of the tracer particle comprises a thiol group (—SH). In some embodiments, the surface of the tracer particle comprises a thioether group (—SR), wherein R is C₁₋₁₂ alkyl. In some embodiments, the surface of the tracer particle comprises an aldehyde group (—CHO). In some embodiments, the surface of the tracer particle comprises an azide group (—N₃). In some embodiments, the surface of the tracer particle comprises an alkyne group (—CH).

As used herein, the “diameter” of a precursor particle is the largest linear dimension of the particle. As used herein, the “diameter” of a tracer particle is the largest linear dimension of the particle.

A precursor particle can have a regular shape (e.g., sphere, tetrahedron, square pyramid, hexagonal pyramid, cube, cuboid, triangular prism, octahedron, pentagonal prism, hexagonal prism, dodecahedron, ellipsoid, icosahedron, cone, or cylinder) or an irregular shape.

A tracer particle can have a regular shape (e.g., sphere, tetrahedron, square pyramid, hexagonal pyramid, cube, cuboid, triangular prism, octahedron, pentagonal prism, hexagonal prism, dodecahedron, ellipsoid, icosahedron, cone, or cylinder) or an irregular shape.

In some embodiments, the tracer particle has a diameter of from about 1 nm to about 1000 nm. In some embodiments, the tracer particle has a diameter of from about 5 nm to about 900 nm. In some embodiments, the tracer particle has a diameter of from about 10 nm to about 800 nm. In some embodiments, the tracer particle has a diameter of from about 25 nm to about 700 nm. In some embodiments, the tracer particle has a diameter of from about 50 nm to about 600 nm. In some embodiments, the tracer particle has a diameter of from about 75 nm to about 500 nm. In some embodiments, the tracer particle has a diameter of from about 100 nm to about 400 nm. In some embodiments, the tracer particle has a diameter of from about 200 nm to about 300 nm.

In some embodiments, the tracer particle has a diameter of from about 1 nm to about 1000 nm. In some embodiments, the tracer particle has a diameter of from about 1 nm to about 900 nm. In some embodiments, the tracer particle has a diameter of from about 1 nm to about 800 nm. In some embodiments, the tracer particle has a diameter of from about 1 nm to about 700 nm. In some embodiments, the tracer particle has a diameter of from about 1 nm to about 600 nm. In some embodiments, the tracer particle has a diameter of from about 1 nm to about 500 nm. In some embodiments, the tracer particle has a diameter of from about 1 nm to about 400 nm. In some embodiments, the tracer particle has a diameter of from about 1 nm to about 300 nm. In some embodiments, the tracer particle has a diameter of from about 1 nm to about 200 nm. In some embodiments, the tracer particle has a diameter of from about 1 nm to about 100 nm.

In some embodiments, the tracer particle has a diameter of about 1000 nm or less. In some embodiments, the tracer particle has a diameter of about 900 nm or less. In some embodiments, the tracer particle has a diameter of about 800 nm or less. In some embodiments, the tracer particle has a diameter of about 700 nm or less. In some embodiments, the tracer particle has a diameter of about 600 nm or less. In some embodiments, the tracer particle has a diameter of about 500 nm or less. In some embodiments, the tracer particle has a diameter of about 400 nm or less. In some embodiments, the tracer particle has a diameter of about 300 nm or less. In some embodiments, the tracer particle has a diameter of about 200 nm or less. In some embodiments, the tracer particle has a diameter of about 100 nm or less. In some embodiments, the tracer particle has a diameter of about 75 nm or less. In some embodiments, the tracer particle has a diameter of about 50 nm or less. In some embodiments, the tracer particle has a diameter of about 25 nm or less. In some embodiments, the tracer particle has a diameter of about 10 nm or less.

In some embodiments, the tracer particle has a diameter of about 1 nm. In some embodiments, the tracer particle has a diameter of about 5 nm. In some embodiments, the tracer particle has a diameter of about 10 nm. In some embodiments, the tracer particle has a diameter of about 25 nm. In some embodiments, the tracer particle has a diameter of about 50 nm. In some embodiments, the tracer particle has a diameter of about 75 nm. In some embodiments, the tracer particle has a diameter of about 100 nm. In some embodiments, the tracer particle has a diameter of about 200 nm. In some embodiments, the tracer particle has a diameter of about 300 nm. In some embodiments, the tracer particle has a diameter of about 400 nm. In some embodiments, the tracer particle has a diameter of about 500 nm. In some embodiments, the tracer particle has a diameter of about 600 nm. In some embodiments, the tracer particle has a diameter of about 700 nm. In some embodiments, the tracer particle has a diameter of about 800 nm. In some embodiments, the tracer particle has a diameter of about 900 nm. In some embodiments, the tracer particle has a diameter of about 1000 nm.

In some embodiments, the plurality of tags comprise at least one member selected from a fluorescent tag, a magnetic tag, an acoustic contrast tag, a thermal tag, a radioactive tag, and a nanoparticle tag. In some embodiments, the plurality of tags comprise a fluorescent tag. In some embodiments, the plurality of tags comprise a magnetic tag. In some embodiments, the plurality of tags comprise an acoustic contrast tag. In some embodiments, the plurality of tags comprise a thermal tag. In some embodiments, the plurality of tags comprise a radioactive tag. In some embodiments, the plurality of tags comprise a nanoparticle tag.

In some embodiments, the plurality of tags are bonded to the precursor particle through chemisorption or physisorption. In some embodiments, the plurality of tags are bonded to the precursor particle through chemisorption. In some embodiments, the plurality of tags are bonded to the precursor particle through physisorption. In some embodiments, the plurality of tags are bonded to the precursor particle through an ionic bond. In some embodiments, the plurality of tags are bonded to the precursor particle through a covalent bond. In some embodiments, the plurality of tags are bonded to the precursor particle through Van der Waals forces. In some embodiments, the plurality of tags are bonded to the precursor particle through dipole-dipole interaction.

In some embodiments, 10% or more of the surface area of the tracer particle is covered by the plurality of tags. In some embodiments, 20% or more of the surface area of the tracer particle is covered by the plurality of tags. In some embodiments, about 30% or more of the surface area of the tracer particle is covered by the plurality of tags. In some embodiments, about 40% or more of the surface area of the tracer particle is covered by the plurality of tags. In some embodiments, about 50% or more of the surface area of the tracer particle is covered by the plurality of tags. In some embodiments, about 60% or more of the surface area of the tracer particle is covered by the plurality of tags. In some embodiments, about 70% or more of the surface area of the tracer particle is covered by the plurality of tags. In some embodiments, about 80% or more of the surface area of the tracer particle is covered by the plurality of tags. In some embodiments, about 90% or more of the surface area of the tracer particle is covered by the plurality of tags.

In some embodiments, the tracer particle further comprises a surfactant. In some embodiments, the surfactant comprises at least one member selected from a nonionic surfactant, a cationic surfactant, an anionic surfactant, and a zwitterionic surfactant. In some embodiments, the surfactant comprises a nonionic surfactant. In some embodiments, the surfactant comprises a cationic surfactant. In some embodiments, the surfactant comprises an anionic surfactant. In some embodiments, the surfactant comprises a zwitterionic surfactant.

In some embodiments, the surfactant comprises at least one member selected from glyceryl monostearate (GMS), sorbitan monostearate (Span 60), poloxamer 188 (Pluronic F68), polysorbate 80 (Tween 80), cetyltrimethylammonium bromide (CTAB), didodecyldimethylammonium bromide (DDAB), sodium dodecyl sulfate (SDS), sodium cholate (SC), oleyl amidopropyl betaine (OAPB), and 3-(N,N-dimethyltetradecylammonio) propane-1-sulfonate (SB3-14). In some embodiments, the surfactant comprises glyceryl monostearate (GMS). In some embodiments, the surfactant comprises sorbitan monostearate (Span 60). In some embodiments, the surfactant comprises poloxamer 188 (Pluronic F68). In some embodiments, the surfactant comprises polysorbate 80 (Tween 80). In some embodiments, the surfactant comprises cetyltrimethylammonium bromide (CTAB). In some embodiments, the surfactant comprises didodecyldimethylammonium bromide (DDAB). In some embodiments, the surfactant comprises sodium dodecyl sulfate (SDS). In some embodiments, the surfactant comprises sodium cholate (SC). In some embodiments, the surfactant comprises oleyl amidopropyl betaine (OAPB). In some embodiments, the surfactant comprises 3-(N,N-dimethyltetradecylammonio) propane-1-sulfonate (SB3-14).

In some embodiments, a concentration of about 50 parts per trillion of tracer particles can be detected from the one or more samples of produced water. In some embodiments, a concentration of at least about 50 parts per trillion of tracer particles can be detected from the one or more samples of produced water.

In some embodiments, the method further comprises plotting the concentration of tracer particles from the one or more samples of produced water as a function of transport time.

In some embodiments, the method further comprises determining the volume of water-filled pores between wells in a petroleum reservoir.

In some embodiments, the method further comprises tracking water flow through a petroleum reservoir.

In some embodiments, the method further comprises determining reservoir heterogeneity.

In some embodiments, the method further comprises evaluating the sweep efficiency of the reservoir.

In some embodiments, the method further comprises detecting leaks in the reservoir.

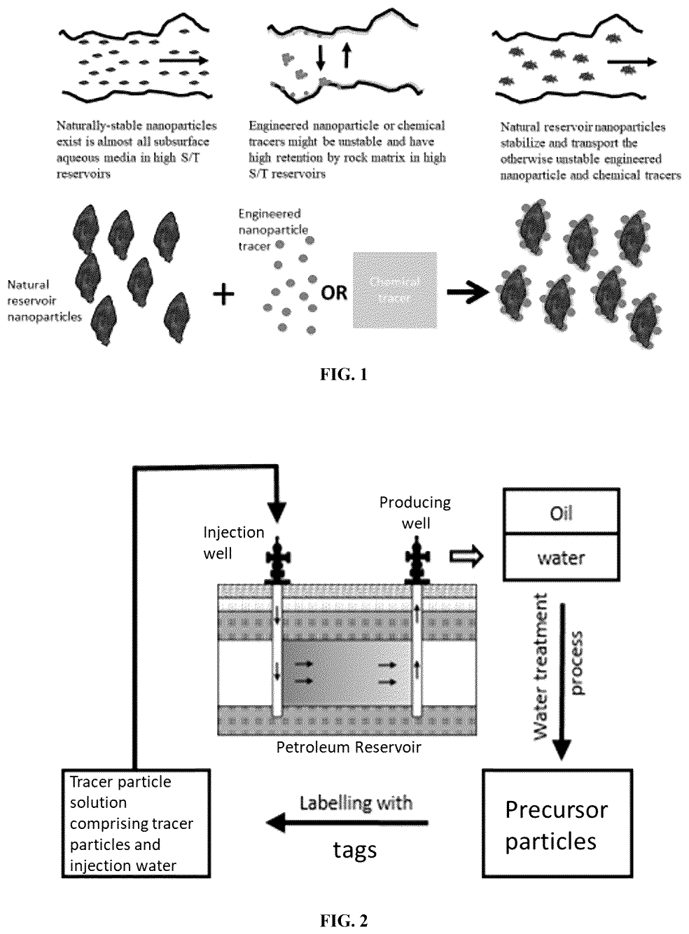

In some embodiments, the method further comprises delivering chemical compounds through the reservoir. FIG. 1 illustrates tracer particles comprising precursor particles isolated from a petroleum reservoir with synthetic tracer particles. Naturally stable nanoparticles (precursor particles) exist in almost all subsurface aqueous media in high salinity, high temperature (high S/T) reservoirs. Engineered nanoparticles or chemical tracers (synthetic tracer particles) may be unstable and have high retention by the rock matrix in high salinity, high temperature (high S/T) reservoirs. Natural reservoir nanoparticles (precursor particles) comprising a plurality of tags (tracer particles) may be more stable and transportable in high salinity, high temperature reservoirs than synthetic tracer particles.

FIG. 2 an exemplary process of isolating precursor particles from a petroleum reservoir; labeling the precursor particle with a plurality of tags; injecting the tracer particles into an injection well of a petroleum reservoir; and collecting a sample of produced water from one or more producing wells. As shown in the upper right hand portion of FIG. 2, precursor particles are isolated from a petroleum reservoir. Referring to the middle bottom portion of FIG. 2, the isolated particles are labeled with one or more tags. The bottom left portion of FIG. 2 shows that the labeled particles are combined with injection water. The upper left portion of FIG. 2 shows that the injection water is injected into an injection well of a petroleum reservoir. The result is produced water that contains the labeled particles, which can be used in a variety of analyses, including, for example, determining the volume of water-filled pores between wells in a petroleum reservoir and tracking water flow through a petroleum reservoir.

### Embodiments

Embodiment 1. A method, comprising:
   isolating a precursor particle from a petroleum reservoir;
   labeling the precursor particle with a plurality of tags to form a tracer particle;
   dispersing a plurality of tracer particles in injection water to form a tracer particle solution;
   injecting the tracer particle solution into an injection well of a petroleum reservoir;
   collecting a sample of produced water from one or more producing wells; and
   measuring the concentration of tracer particles from the one or more samples of produced water.
Embodiment 2. The method of embodiment 1, wherein the precursor particle is isolated from produced water from a petroleum reservoir.
Embodiment 3. The method of embodiment 1 or 2, wherein the precursor particle is isolated from the petroleum reservoir by ultrafiltration or cross-flow filtration.
Embodiment 4. The method of any one of embodiments 1-3, wherein the precursor particle comprises at least one member selected from the group consisting of clay and mineral fragments.
Embodiment 5. The method of any one of embodiments 1-4, wherein the surface of the tracer particle comprises at least one member selected from a polyethylene glycol (PEG), an antibody, a peptide, an organic dye, a paramagnetic functional group, and a super magnetic functional group.
Embodiment 6. The method of any one of embodiments 1-4, wherein the surface of the tracer particle comprises at least one member selected from the group consisting of an amino group (—NH₂), an alkylamino group (—NHR), a dialkylamino group (—NR₂), a carboxy group (—COOH), a hydroxy group (—OH), a thiol group (—SH), a thioether group (—SR), an aldehyde group (—CHO), an azide group (—N₃), and an alkyne group (—CH), wherein each R is C₁₋₁₂ alkyl.
Embodiment 7. The method of any one of embodiments 1-6, wherein the tracer particle has a diameter of from 1 nm to 1000 nm.
Embodiment 8. The method of any one of embodiments 1-6, wherein the tracer particle has a diameter of from 1 nm to 100 nm.
Embodiment 9. The method of any one of embodiments 1-8, wherein the plurality of tags comprise at least one member selected from the group consisting of a fluorescent tag, a magnetic tag, an acoustic contrast tag, a thermal tag, a radioactive tag, and a nanoparticle tag.
Embodiment 10. The method of any one of embodiments 1-9, wherein the plurality of tags are bonded to the precursor particle through chemisorption or physisorption.
Embodiment 11. The method of embodiment 10, wherein the plurality of tags are bonded to the precursor particle through chemisorption, wherein the chemisorption is an ionic bond or a covalent bond.
Embodiment 12. The method of embodiment 10, wherein the plurality of tags are bonded to the precursor particle through physisorption, wherein the physisorption is Van der Waals forces or dipole-dipole interaction.
Embodiment 13. The method of any one of embodiments 1-12, wherein 10% or more of the surface area of the tracer particle is covered by the plurality of tags.
Embodiment 14. The method of any one of embodiments 1-12, wherein 20% or more of the surface area of the tracer particle is covered by the plurality of tags.
Embodiment 15. The method of any one of embodiments 1-14, wherein the tracer particle further comprises a surfactant.
Embodiment 16. The method of embodiment 15, wherein the surfactant comprises at least one member selected from the group consisting of a nonionic surfactant, a cationic surfactant, an anionic surfactant, and a zwitterionic surfactant.
Embodiment 17. The method of embodiment 15, wherein the surfactant comprises at least one member selected from the group consisting of glyceryl monostearate (GMS), sorbitan monostearate (Span 60), poloxamer 188 (Pluronic F68), polysorbate 80 (Tween 80), cetyltrimethylammonium bromide (CTAB), didodecyldimethylammonium bromide (DDAB), sodium dodecyl sulfate (SDS), sodium cholate (SC), oleyl amidopropyl betaine (OAPB), and 3-(N,N-dimethyltetradecylammonio) propane-1-sulfonate (SB3-14).
Embodiment 18. The method of any one of embodiments 1-17, wherein a concentration of 50 parts per trillion of tracer particles can be detected from the one or more samples of produced water.
Embodiment 19. The method of any one of embodiments 1-18, further comprising:
   plotting the concentration of tracer particles from the one or more samples of produced water as a function of transport time.
Embodiment 20. The method of embodiment 19, further comprising:
   determining the volume of water-filled pores between wells in a petroleum reservoir.
Embodiment 21. The method of embodiment 19, further comprising:
   tracking water flow through a petroleum reservoir.
Embodiment 22. The method of embodiment 19, further comprising:
   determining reservoir heterogeneity.
Embodiment 23. The method of embodiment 19, further comprising:
   evaluating the sweep efficiency of the reservoir.
Embodiment 24. The method of embodiment 19, further comprising:
   detecting leaks in the reservoir.

### EXAMPLES

### Example 1-Preparation and Use of Tracer Particles

Natural colloidal and suspended particles (i.e., precursor particles) are extracted from petroleum reservoirs using ultrafiltration, cross-flow filtration, or any other suitable method, and concentrated. Preferred precursor particles have sizes between about 1 and about 1000 nm. The isolated and concentrated precursor particles are cleaned to remove background contamination, and further concentrated, as needed. The precursor particles are labeled with chemical and/or nanoparticle tags to form tracer particles.

The tracer particles are dispersed in injection water and injected into an injection well. Samples of produced water are collected (see FIG. 2). Detection systems are used to analyze and determine the concentration of the tracer particles in the sample of produced water as a function of transport time. The determined concentration is used to determine: (a) the volume of water-filled pores between wells in a petroleum reservoir; (b) water flow through a petroleum reservoir; (c) reservoir heterogeneity; (d) the sweep efficiency of the reservoir; and/or (e) the presence of leaks in the reservoir.

What is claimed:

1. A method, comprising:
isolating a precursor particle from a petroleum reservoir;
labeling the precursor particle with a plurality of tags to form a tracer particle;
dispersing a plurality of tracer particles in injection water to form a tracer particle solution;
injecting the tracer particle solution into an injection well of a petroleum reservoir;
collecting a sample of produced water from one or more producing wells; and
measuring the concentration of tracer particles from the one or more samples of produced water.

2. The method of claim 1, wherein the precursor particle is isolated from produced water from a petroleum reservoir.

3. The method of claim 1, wherein the precursor particle is isolated from the petroleum reservoir by ultrafiltration or cross-flow filtration.

4. The method of claim 1, wherein the precursor particle comprises at least one member selected from the group consisting of clay and mineral fragments.

5. The method of claim 1, wherein the surface of the tracer particle comprises at least one member selected from the group consisting of an amino group (—NH₂), an alkylamino group (—NHR), a dialkylamino group (—NR₂), a carboxy group (—COOH), a hydroxy group (—OH), a thiol group (—SH), a thioether group (—SR), an aldehyde group (—CHO), an azide group (—N₃), and an alkyne group (—CH), wherein each R is C₁₋₁₂ alkyl.

6. The method of claim 1, wherein the tracer particle has a diameter of from 1 nm to 1000 nm.

7. The method of claim 1, wherein the tracer particle has a diameter of from 1 nm to 100 nm.

8. The method of claim 1, wherein the plurality of tags comprise at least one member selected from the group consisting of a chemical fluorescent tag, a nanoparticle fluorescent tag, a chemical magnetic tag, a nanoparticle magnetic tag, a chemical acoustic contrast tag, a nanoparticle acoustic contrast tag, a chemical thermal tag, a nanoparticle thermal tag, a chemical radioactive tag, and a nanoparticle radioactive tag.

9. The method of claim 1, wherein the plurality of tags are bonded to the precursor particle through chemisorption or physisorption.

10. The method of claim 9, wherein the plurality of tags are bonded to the precursor particle through chemisorption, wherein the chemisorption is an ionic bond or a covalent bond.

11. The method of claim 9, wherein the plurality of tags are bonded to the precursor particle through physisorption, wherein the physisorption is Van der Waals forces or dipole-dipole interaction.

12. The method of claim 1, wherein 10% or more of the surface area of the tracer particle is covered by the plurality of tags.

13. The method of claim 1, wherein 20% or more of the surface area of the tracer particle is covered by the plurality of tags.

14. The method of claim 1, wherein the tracer particle further comprises a surfactant.

15. The method of claim 14, wherein the surfactant comprises at least one member selected from the group consisting of a nonionic surfactant, a cationic surfactant, an anionic surfactant, and a zwitterionic surfactant.

16. The method of claim 14, wherein the surfactant comprises at least one member selected from the group consisting of glyceryl monostearate (GMS), sorbitan monostearate (Span 60), poloxamer 188 (Pluronic F68), polysorbate 80 (Tween 80), cetyltrimethylammonium bromide (CTAB), didodecyldimethylammonium bromide (DDAB), sodium dodecyl sulfate (SDS), sodium cholate (SC), oleyl amidopropyl betaine (OAPB), and 3-(N,N-dimethyltetradecylammonio) propane-1-sulfonate (SB3-14).

17. The method of claim 1, wherein a concentration of 50 parts per trillion of tracer particles can be detected from the one or more samples of produced water.

18. The method of claim 1, further comprising:
plotting the concentration of tracer particles from the one or more samples of produced water as a function of transport time.

19. The method of claim 18, further comprising determining the volume of water-filled pores between wells in a petroleum reservoir.

20. The method of claim 1, further comprising:
tracking water flow through a petroleum reservoir;
determining reservoir heterogeneity;
evaluating the sweep efficiency of the reservoir; or
detecting leaks in the reservoir.
